(19) United States
(12) Patent Application Publication
(10) Pub. No.: US 2015/0073239 A1
(43) Pub. Date: Mar. 12, 2015

(54) CONTINUOUS CUFFLESS BLOOD PRESSURE MEASUREMENT USING A MOBILE DEVICE

(71) Applicant: Maxim Integrated Products, Inc., San Josse, CA (US)

(72) Inventors: Cheng-Wei Pei, Belmont, CA (US); Chirag C. Sthalekar, Medford, MA (US)

(73) Assignee: Maxim Integrated Products, Inc., San Jose, CA (US)

(21) Appl. No.: 14/265,434

(22) Filed: Apr. 30, 2014

Related U.S. Application Data
(60) Provisional application No. 61/875,211, filed on Sep. 9, 2013.

(51) Int. Cl. A61B 5/00; A61B 5/021; A61B 7/04; A61B 5/1455

(52) U.S. Cl. CPC A61B 5/6898; A61B 5/14551; A61B 5/02125; A61B 7/04; A61B 5/742
USPC 600/324; 600/480

(57) ABSTRACT

A mobile blood pressure monitor is described that includes an integrated acoustic device, an optical sensor including at least one of a light source or a pulse oximeter device, and control circuitry coupled to the integrated acoustic device and the optical sensor. Additionally, a mobile electronic device configured to measure blood pressure is described that includes a mobile system and a mobile blood pressure monitor as disclosed above. In implementations, a process for measuring blood pressure includes sensing a heart sound with an integrated acoustic device, measuring a blood pulse rate at a peripheral site with an optical sensor, calculating a pulse wave transit time using a sensed heart sound and a measured blood pulse rate, and correlating a blood pressure using the heart sound and the blood pulse rate.

### BACKGROUND

Blood pressure, sometimes referred to as arterial blood pressure, is the pressure exerted by circulating blood upon the walls of blood vessels and is one of the principal vital signs. During each heartbeat, blood pressure varies between a maximum (systolic) and a minimum (diastolic) pressure. The blood pressure in the circulation is principally due to the pumping action of the heart. Differences in mean blood pressure are responsible for blood flow from one location to another in the circulation. The rate of mean blood flow depends on the resistance to flow presented by the blood vessels. Mean blood pressure decreases as the circulating blood moves away from the heart through arteries and capillaries due to viscous losses of energy.

### SUMMARY

A mobile blood pressure monitor is described that includes an integrated acoustic device, an optical sensor including at least one of a light source or a pulse oximeter device, and control circuitry coupled to the integrated acoustic device and the optical sensor. Additionally, a mobile electronic device configured to measure blood pressure is described that includes a mobile system and a mobile blood pressure monitor as disclosed above. In implementations, a process for measuring blood pressure includes sensing a heart sound with an integrated acoustic device, measuring a blood pulse rate at a peripheral site with an optical sensor, calculating a pulse wave transit time using a sensed heart sound and a measured blood pulse rate, and correlating a blood pressure using the heart sound and the blood pulse rate.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The detailed description is described with reference to the accompanying figures. The use of the same reference numbers in different instances in the description and the figures may indicate similar or identical items.

FIG. 1A is an isometric front view illustrating a mobile blood pressure monitor in accordance with example implementations of the present disclosure.

FIG. 1B is an isometric rear view illustrating a mobile blood pressure monitor in accordance with example implementations of the present disclosure.

FIG. 1C is a partial bottom view illustrating a mobile blood pressure monitor in accordance with example implementations of the present disclosure.

FIG. 1D is a graphical illustration of a heart sound measurement and a blood pulse rate using a mobile blood pressure monitor in accordance with example implementations of the present disclosure.

FIG. 1E is an environmental illustration of a mobile blood pressure device and a mobile blood pressure monitor in accordance with example implementations of the present disclosure.

FIG. 2 is a flow diagram illustrating a process in an example implementation for utilizing a mobile blood pressure monitor, such as the mobile blood pressure monitors shown in FIGS. 1A through 1E.

### DETAILED DESCRIPTION

### Overview

Current solutions for measuring blood pressure often include using a traditional sphygmomanometer based measurement that uses a blood pressure cuff. Other solutions include using body contact sensors, such as in the case of an electrocardiogram device. Non-invasive and non-occlusive blood pressure measurement can include sensing two physiological parameters concurrently. However, using invasive or occlusive methods and/or multiple devices is inconvenient and undesirable.

Accordingly, a mobile blood pressure monitor is described that includes an integrated acoustic device, an optical sensor including at least one of a light source or a pulse oximeter device, and control circuitry coupled to the integrated acoustic device and the optical sensor. Additionally, a mobile electronic device configured to measure blood pressure is described that includes a mobile system and a mobile blood pressure monitor as disclosed above. In implementations, a process for measuring blood pressure includes sensing a heart sound with an integrated acoustic device, measuring a blood pulse rate at a peripheral site with an optical sensor, calculating a pulse wave transit time using a sensed heart sound and a measured blood pulse rate, and correlating a blood pressure using the heart sound and the blood pulse rate.

### Example Implementations

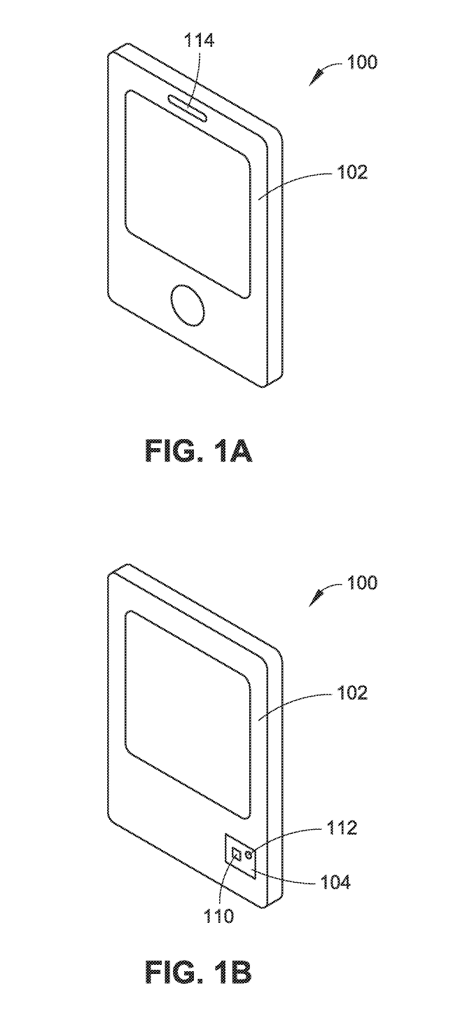

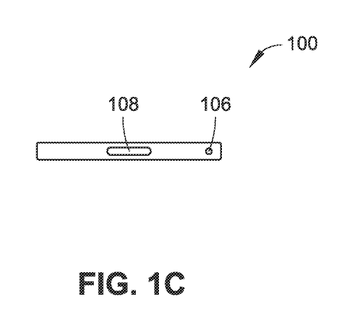

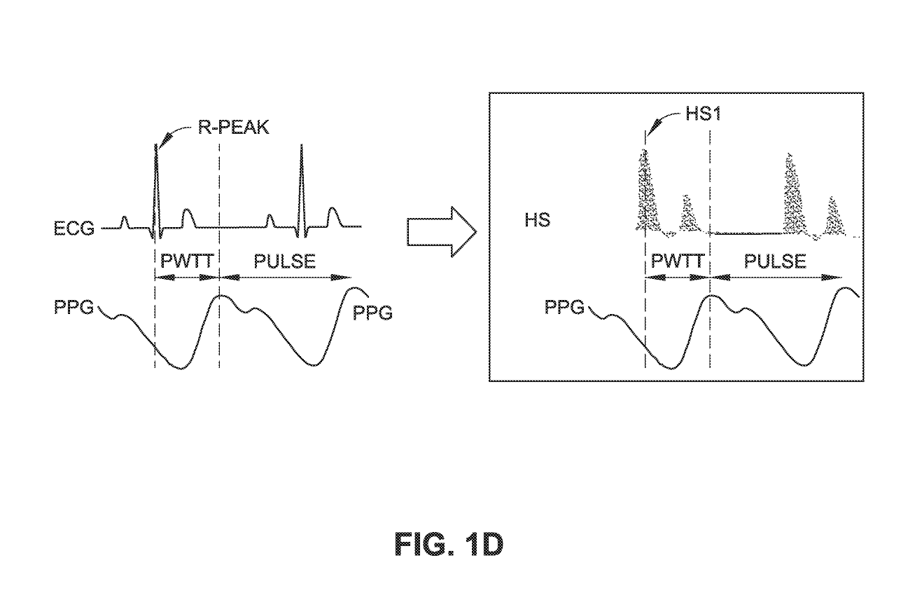

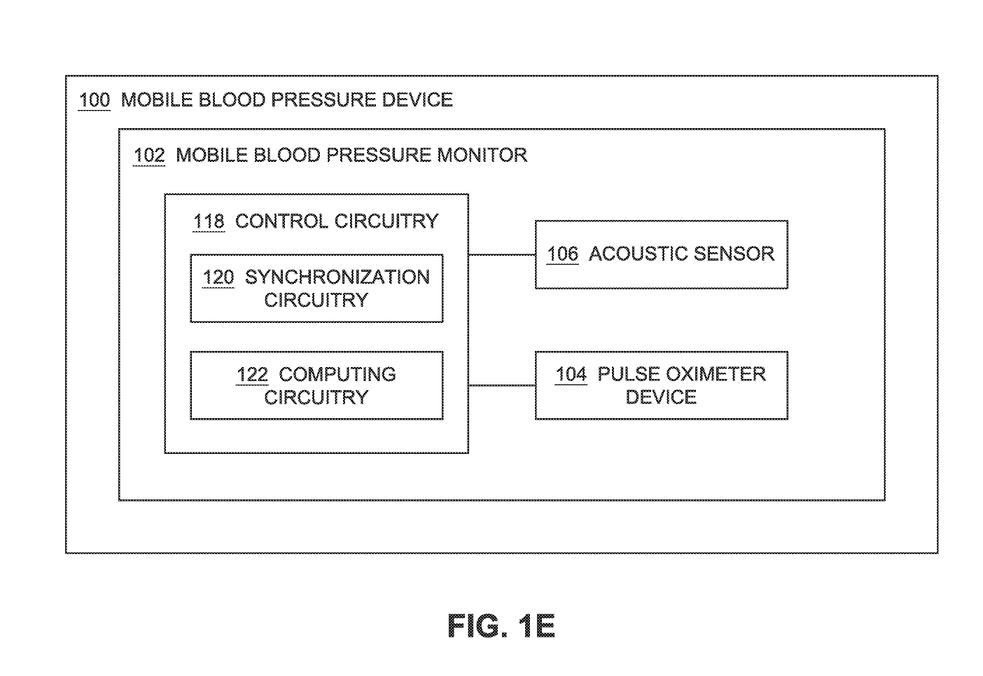

FIGS. 1A through 1E illustrate a mobile blood pressure monitor 102 and mobile blood pressure device 100 in accordance with example implementations of the present disclosure. As shown, the mobile blood pressure monitor 102 includes an integrated acoustic device 106. The integrated acoustic device 106 may include a microphone configured to be disposed as a component in a cell phone, smartphone, or other device, and may include a device configured to detect sound. In implementations, the integrated acoustic device 106 can include a microphone that is used as the microphone portion of a smartphone (e.g., the same microphone used when a person uses the device as a phone and speaks into). In another implementation, the integrated acoustic device 106 can include a second microphone. The integrated acoustic device 106 may include an acoustic-to-electric transducer and/or sensor that can be configured to convert a heart sound into an electric signal. Some examples of microphones that can be used as an integrated acoustic device 106 can include a condenser microphone, a dynamic microphone, an electret condenser microphone, a ribbon microphone, a carbon microphone, a piezoelectric microphone, a fiber optic microphone, a laser microphone, a liquid microphone, and/or a MEMS microphone. The integrated acoustic device 106 may be placed in different locations of the mobile blood pressure device 100. In one embodiment and as shown in FIG. 3, the integrated acoustic device 106 can be placed at the bottom of the mobile blood pressure device 100 (e.g., proximate to a charging connector 108).

In some implementations, the mobile blood pressure monitor 102 can include an optical sensor 104 including at least one of a light source 112 or a pulse oximeter device 110. A pulse oximeter device 110 can include a medical device that indirectly monitors the oxygen saturation of a patient's blood (as opposed to measuring oxygen saturation directly through a blood sample) and changes in blood volume in the skin, producing a photoplethysmogram. An optical sensor 104 may be incorporated into a health monitor, such as the mobile blood pressure device 100 and/or mobile blood pressure monitor 102. In other implementations, the pulse oximeter device 104 can include any device capable of detecting a photoplethysmogram (PPG) signal.

The optical sensor 104 can include a light source 112 and a pulse oximeter device 110 or other detector (e.g., photodiode). In some implementations, the light source 112 can include at least one small light-emitting diode (LED) and a pulse oximeter device 110 (e.g., photodiode) through a translucent part of the patient's body (e.g., a fingertip, an earlobe, etc.). In other implementations, the light source can include a laser. In one implementation, the optical sensor 104 (e.g., both the light source 112 and the pulse oximeter device 110) is disposed on the back (e.g., the side distal from a display and/or speaker 114) of the mobile blood pressure device 100. In this implementation, the light source 112 and the pulse oximeter device 110 can, but are not required, to face each other. This configuration can allow for user convenience while holding the mobile blood pressure device 100. In these implementations, when a LED is used, one LED can be red, with a wavelength of 660 nm, for example, and another can be infrared (e.g., 905, 910, or 940 nm). The wavelength range can include about 400 nm through about 1000 nm. Absorption at these wavelengths differs significantly between oxyhemoglobin and its deoxygenated form. Therefore, the oxy/deoxyhemoglobin ratio can be calculated from the ratio of the absorption of the red and infrared light. The absorbance of oxyhemoglobin and deoxyhemoglobin is the same (the isosbestic point) for the wavelengths of 590 and 805 nm. The monitored signal fluctuates in time with the heart beat because the arterial blood vessels expand and contract with each heartbeat. Thus, detecting a pulse is essential to the operation of a pulse oximeter and it will not function without a pulse.

The mobile blood pressure device 100 includes control circuitry 118. In implementations, control circuitry 118 can include hardware, software, and/or firmware configured to correlate blood pressure using an optical sensor 104 and an integrated acoustic device 106. In an implementation, control circuitry 118 includes computing circuitry 122 (e.g., a computer processor and memory) with instructions for determining and/or correlating blood pressure from measurements (e.g., collected waveforms) received from the optical sensor 104 and the integrated acoustic device 106. In embodiments, the collected waveforms may be processed with the computer processor using backend software and can be displayed on a suitable frontend software application.

Non-invasive and non-occlusive blood pressure measurement can include sensing two physiological parameters, which can include two timing measurements of an individual's pulse across a known distance. The mobile blood pressure device 100 can include an integrated acoustic device 106 and optical sensor 104 integrated onto a mobile device (e.g., mobile blood pressure device 100) to measure the systolic blood pressure (SBP) using the pulse wave transit time (PWTT). Blood pressure can be empirically measured and accepted to be related to the time of arrival of a pulse between the aortic valve and a peripheral site (e.g., such as a finger). As shown in FIG. 1D, this method can include the measurement of the time difference between the ‘R’ peak in an ECG waveform and the peak of the pulse measured using photoplethysmography (PPG) at the peripheral site (e.g., a finger). Heart sounds (e.g., HS1 in FIG. 1D) can be measured using a sensitive microphone (e.g., integrated acoustic device 106) and an optical PPG sensor (e.g., pulse oximeter device 110) that are integral to a mobile blood pressure device 100 (e.g., mobile phone, smartphone, etc.) for measuring the blood pressure. The heart sounds characterized by 4 typical sounds (e.g., HS1, HS2, HS3, HS4) can be correlated with specific peaks in an ECG waveform. The characteristic HS1 sound corresponds to the ‘R’ peak in the ECG waveform and can be detected easily using the integrated acoustic device 106 on the phone. In a specific embodiment, the pulse is measured by placing the finger on an optical sensor 104 that has a light source 112 that transmits light into the tissue and a pulse oximeter device 110 that detects the reflected light. The blood flow in the arteries of the finger causes pulsation of the light at the pulse oximeter device 110. The pulsation relates to the pulse rate of the subject at the periphery. Use of the heart sound(s) to measure blood pressure can replace the requirement of an ECG measurement.

### Example Processes

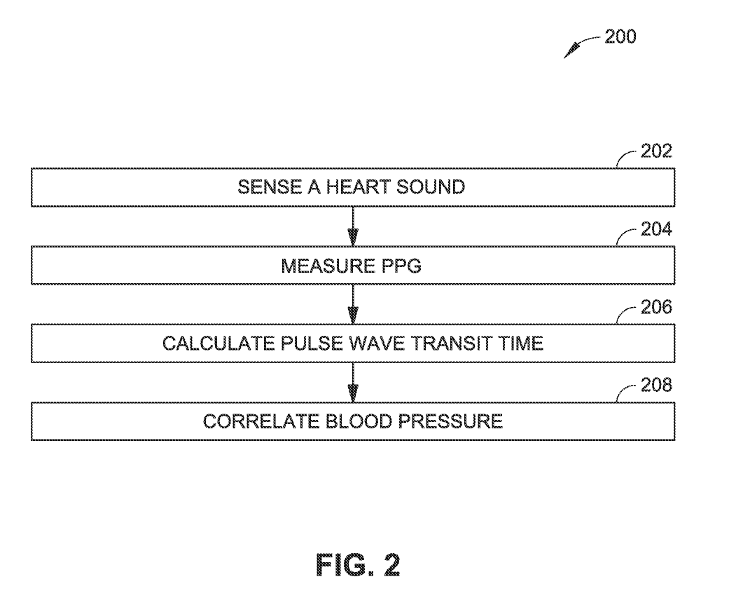

FIG. 2 illustrates an example process 200 that employs blood pressure measurement techniques using a blood pressure device, such as the mobile blood pressure device 100 shown in FIGS. 1A through 1E.

As shown in FIG. 2, a heart sound is sensed (Block 202). In an implementation, an integrated acoustic device 106 disposed as a portion of the mobile blood pressure device 100 can be placed proximate to an individual's heart. Placing the integrated acoustic device 106 proximate to the heart allows heart sounds (e.g., a heartbeat) to be detected. The heart sounds may correlate to specific peaks in an ECG waveform using control circuitry 118 and/or a microprocessor.

A blood pulse rate is measured (Block 204). In an implementation, measuring the blood pulse rate includes placing a finger (or other peripheral site) on an optical sensor 104, (e.g., pulse oximeter device) with a light source 112 that transmits light into the finger tissue, and the pulse oximeter device 110 detects the light reflected from the finger to the pulse oximeter device 110.

A pulse wave transit time is calculated (Block 206). In an implementation, calculating a pulse wave transit time can include using synchronization circuitry 120 and/or computing circuitry 122. As illustrated in FIGS. 1D and 1E, calculating the pulse wave transit time can include using computing circuitry 122 for calculating the time difference between an R-peak of an ECG and the peak of a PPG. In FIG. 1D, heart sound 1 (HS1) corresponds with the illustrated R-peak.

A blood pressure is correlated using a blood pulse rate (Block 208). In an implementation, correlating a blood pressure can include using a blood pulse rate, a heart sound, and/or control circuitry 118. The time of arrival of a pulse between the aortic valve and the peripheral site can be correlated with specific peaks in a waveform determined from the blood pulse rate and the heart sound to determine an individual's blood pressure. In a specific embodiment, computing circuitry 122 can use a measured pulse wave transit time to correlate with empirical data from a database, for example. Empirical data may be obtained from sources, such as online databases, memory that is included in control circuitry 118, etc.

### CONCLUSION

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. A mobile blood pressure monitor, comprising:
an integrated acoustic device;
an optical sensor; and
control circuitry coupled to the integrated acoustic device and the optical sensor.

2. The mobile blood pressure monitor of claim 1, wherein the integrated acoustic device includes a microphone sensor.

3. The mobile blood pressure monitor of claim 1, wherein the integrated acoustic device is disposed on a first side of the mobile blood pressure monitor that is adjacent to a second side of the mobile blood pressure monitor with the optical sensor device.

4. The mobile blood pressure monitor of claim 1, wherein the optical sensor includes a pulse oximeter device.

5. The mobile blood pressure monitor of claim 1, wherein the control circuitry is configured to measure pulse wave transit time.

6. The mobile blood pressure monitor of claim 1, wherein the control circuitry includes synchronization circuitry configured to synchronize a heart sound measurement and a photoplethysmography (PPG) measurement.

7. The mobile blood pressure monitor of claim 1, wherein the control circuitry includes computing circuitry configured to control the integrated acoustic device and the pulse oximeter device.

8. The mobile blood pressure monitor of claim 1, wherein the optical sensor includes a light source.

9. A mobile electronic device configured to measure blood pressure, comprising:
a mobile system; and
a mobile blood pressure monitor integrated into the mobile system, including
an integrated acoustic device;
an optical sensor; and
control circuitry coupled to the integrated acoustic device and the optical sensor.

10. The mobile electronic device of claim 9, wherein the mobile system includes a cell phone device.

11. The mobile electronic device of claim 9, wherein the mobile system includes a smart phone device.

12. The mobile electronic device of claim 9, wherein the mobile system includes a tablet computing device.

13. The mobile electronic device of claim 9, wherein the acoustic device includes a microphone sensor.

14. The mobile electronic device of claim 9, wherein the acoustic device is disposed on a first side of the mobile electronic device that is adjacent to a second side of the mobile electronic device with the optical sensor.

15. The mobile electronic device of claim 9, wherein the optical sensor includes a pulse oximeter device.

16. The mobile electronic device of claim 9, wherein the optical sensor includes a light source.

17. A process for measuring blood pressure, comprising:
sensing a heart sound with an integrated acoustic device;
measuring a blood pulse rate at a peripheral site with an optical sensor;
calculating a pulse wave transit time using a sensed heart sound and a measured blood pulse rate; and
correlating a blood pressure using the heart sound, the blood pulse rate, and the pulse wave transit time.

18. The process for measuring blood pressure of claim 17, wherein measuring a blood pulse rate includes measuring a photoplethysmography (PPG) measurement.

19. The process for measuring blood pressure of claim 17, wherein measuring a blood pulse rate at a peripheral site includes measuring the blood pulse rate from a finger.

20. The process for measuring blood pressure of claim 17, wherein correlating the blood pressure includes correlating a systolic blood pressure.
